(19) United States
(12) Patent Application Publication
(10) Pub. No.: US 2013/0004651 A1
(43) Pub. Date: Jan. 3, 2013

(54) SUSTAINED DRUG RELEASE FROM BODY IMPLANTS USING NANOPARTICLE-EMBEDDED POLYMERIC COATING MATERIALS

(75) Inventor: Patty Fu-Giles, University Park, IL (US)

(73) Assignee: Patty Fu-Giles, University Park, IL (US)

(21) Appl. No.: 13/560,730

(22) Filed: Jul. 27, 2012

Related U.S. Application Data
(63) Continuation-in-part of application No. 13/574,033.

(51) Int. Cl. A61K 9/50; A61P 31/04; B05D 7/00; A61P 27/06; A61P 25/00; A61P 39/06; A61K 31/165; A61P 29/00

(52) U.S. Cl. 427/2.26; 424/490; 424/494; 424/497; 514/628; 427/2.24

(57) ABSTRACT

The present invention relates to the preparation of therapeutic compositions including drug-containing nanoparticles for coating a body implant to provide for drug delivery in a locally applied and extended release manner and their methods of use to treat physiological conditions.

### CROSS-REFERENCE TO RELATED APPLICATIONS

This application claims priority from U.S. Provisional Application Ser. No. 61/360,802, filed Jul. 1, 2010 and PCT Application No. PCT/US2011/42776, filed Jul. 1, 2011 and is a continuation-in-part of U.S. Ser. No. 13/574,033, filed Jul. 19, 2012, the disclosures of which are incorporated herein by reference.

### TECHNICAL FIELD

The present invention relates to the sustained release of encapsulated antibiotics and other drugs from a polymeric coating material and, in particular, to a nanoparticulate system for delivering antibiotics/drugs in a locally applied and extended release manner to patients receiving a body implant.

### BACKGROUND OF THE INVENTION

It has been shown that drug delivery systems using nanoparticle-encapsulated antibiotics can improve antimicrobial efficacy against drug-resistant strains. (Torchilin, 2001; Nandi et al., 2003 Garay-Jimenez et al., 2009). Nanoparticles such as liposomes and micelles have been used to protect drugs within a relatively impermeable bilayer or multilayer environment and to prolong release times by isolating the encapsulated drugs from systematic degrading enzymes. Liposomes, micelles and other nanoparticles can be taken up by cells without overt cytotoxic effects, thus enhancing the cellular uptake of the encapsulated material and promoting diffusion across the bacterial or viral envelope. (Torchilin, 2001; Muller-Goymann, 2004; Wang, 2009). Moreover, such nanoparticles are natural, biodegradable and non-toxic.

The major focus of nanoparticulate drug delivery systems to date has related to nanoparticles as polymeric carriers for anticancer agents or for gene delivery and tissue engineering. (Henry, 2002; Richter, 2010). There is an advantage to providing antibiotics and other drugs in the form of nanoparticles to provide for prolonged release in treating infection. Thus, there is a need for a system including antibiotics and other drugs encapsulated within liposomes, micelles and other nanoparticles to treat and alleviate post-surgical and post-transplantation infections, particularly in reaction to body implants.

### SUMMARY OF THE INVENTION

The present invention relates to a nanoparticulate system for delivering antibiotics and other drugs in a locally applied and extended-release manner to patients receiving a body implant. In a preferred embodiment, the body implant comprises an ophthalmic device including an ocular implant for application to the posterior portion of the eye, a glaucoma shunt device or stent, an intrascleral implant or an implantable miniature telescope. Other body implants according to the present invention include dental implants, cochlear implants, nasal implants, and implanted prostheses including vascular grafts, stents, and devices for hip, shoulder and knee replacement.

The method of the present invention, with reference to antibiotic delivery, includes: (1) encapsulating a hydrophobic antibiotic (for example, fluoroquinolone, chloramphenicol and/or rifampicin) and/or a hydrophilic antibiotic (for example, vancomycin and acyclovir) into antibiotic-containing nanoparticles; (2) incorporating the antibiotic-containing nanoparticles into a polymeric coating material (for example, Poloxamer, hydroxypropylmethylcellulose, methylcellulose, polyvinyl alcohol and polyvinyl pyrrolidone) with a volatile carrier solvent (ethyl acetate or ethanol); and (3) applying the product of step (2) to an implant before surgery.

When the volatile carrier solvent evaporates, the polymeric coating material with embedded antibiotic nanoparticles forms a thin film that attaches to the surface of the implant. Local application of encapsulated antibiotics directly to an implant or surgical site provides a non-oral, non-intravenous, controlled time-release method for providing continuous administration of an antibiotic over a prescribed time period. The invention provides a novel chemotherapeutic approach in more efficient, effective doses for the prevention and treatment of bacterial, fungal and viral infections that are often associated with implants.

Examples of antibiotics suitable for use in ophthalmic devices include amoxicillin, sulfa drugs, erythromycin, streptomycin, tetracycline, clarithromycin, terconazole, azithromycin, bacitracin, ciprofloxacin, evofloxacin, ofloxacin, levofloxacin, moxfloxicin, gatifloxacin, aminoglycosides, tobramycin, gentamicin and polymyxin B combinations including polymyxin B/trimethoprim, polymyxin B/bacitracin and polymyxin B/neomycin/gramicidin, including pharmacologically acceptable salts and acids thereof.

Depending on the body implant, other suitable antibiotics include rifampicin, chloramphenicol, novobiocin, spectinomycin, trimethoprim, erythromycin, doxycycline, minocycline, vancomycin, acyclovir, amphotericin B, gentamicin, gentamicin sulfate, tobramycin, ampicillin, penicillin, ethambutol, clindamycin, and cephalosporins including cefazolin, ceftriaxone and cefotaxime, including pharmacologically acceptable salts and acids thereof.

Other suitable drugs, as further described herein, include steroids, anti-inflammatories, glaucoma treatment compounds, anti-histamines, dry eye medication, neuroprotectives, retinoids, antineovasculars, antioxidants and biologics.

An advantage of the present invention is the development of a novel nanovesicular drug delivery system that offers improved pharmaceutical properties, is easily integrated onto the surface of body implant prior to surgery, and facilitates the delivery of drugs/antibiotics to treat a physiological condition, and in the case of antibiotics, to prevent post-operative infections.

This specific targeting drug delivery system helps reduce undesired side effects. It also eliminates the time that otherwise is needed for the drugs to be processed by the liver. Therefore, a reduced amount of the drug will produce comparable beneficiary effects compared to the amount of drug usually required for intravenous or oral administration.

Furthermore, the present delivery system can be customized based on the needs of the patient by varying the entrapped antibiotics/drugs and the mixture of nanostructures in the drug delivery assay. Finally, all nanovesicles in this system are composed of organic materials, which are already used in many FDA-approved drug delivery systems.

### BRIEF DESCRIPTION OF THE DRAWINGS

FIG. 1 is a partial cutaway view of a liposome with a double membrane that encapsulates hydrophilic molecules in its core and hydrophobic molecules in its lipid bilayer in aqueous solution;

FIG. 2 is a partial cutaway view of a micelle with a hydrophobic core and a hydrophilic outer layer or shell which allows the encapsulation of hydrophobic molecules in an aqueous solution;

FIG. 3 is a transmission electron microscopy (TEM) image of encapsulated rifampicin nanoparticles, the lower image showing no aggregation of the nanoparticles within the matrix;

FIG. 4 shows a fluorescent spectrum before and after encapsulation;

FIG. 5 includes data from TEM Dynamic Light Scattering analyses demonstrating nanoparticle average size, uniform nanoparticle size distribution and lack of nanoparticle contamination; and

FIG. 6 includes graphs showing the sustained release of a drug from liposomes and reverse micelles over time.

### DETAILED DESCRIPTION OF THE INVENTION

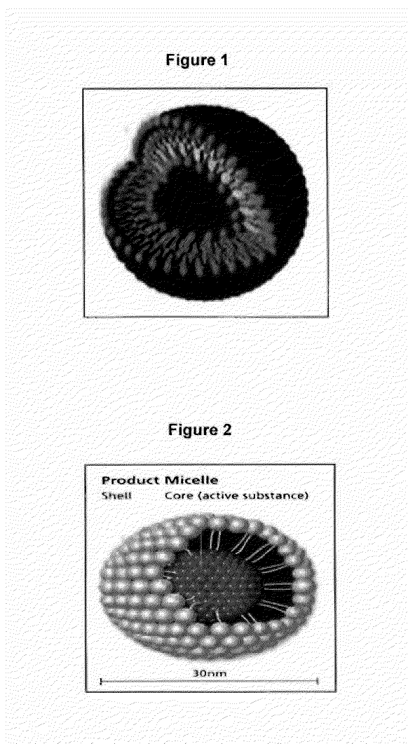

FIG. 1 shows a liposome with a double membrane that can encapsulate both hydrophilic molecules in its core and hydrophobic molecules in the lipid bilayer in an aqueous solution. Liposomes are closed lipid bilayer membranes containing an entrapped aqueous volume. Liposomes can comprise unilamellar vesicles with a single membrane bilayer or multilamellar vesicles including onion-like structures with multiple membrane bilayers, each separated from the next by an aqueous layer. The bilayer comprises two lipid monolayers including a hydrophobic tail region and a hydrophilic head region. The structure of the membrane bilayer is such that the hydrophobic (nonpolar) tails of the lipid monolayers orient towards the center of the bilayer while the hydrophilic heads orient towards the aqueous phase.

The original liposome preparation of Bangham et al. (J. Mol. Biol., 1965, 13:238-252) involves suspending phospholipids in an organic solvent which is then evaporated to dryness leaving a phospholipid film on the reaction vessel. An appropriate amount of aqueous phase is then added, the mixture is allowed to “swell,” and the resulting liposomes which comprise multilamellar vesicles (MLVs) are dispersed by mechanical means. This technique provides the basis for the development of the small sonicated unilamellar vesicles described by Papahadjopoulos et al. (Biochem. Biophys. Acta., 1967, 135:624-638), and large unilamellar vesicles.

As shown in FIG. 2, a typical micelle has a hydrophobic core and a hydrophilic outer surface or shell allowing the encapsulation of hydrophobic molecules in an aqueous solution. A typical micelle in aqueous solution forms an aggregate with the hydrophilic head regions in contact with the surrounding solvent, entrapping the hydrophobic tail regions in the micelle center. The difficulty of filling all the volume of the interior of the bilayer, while accommodating the area per head group forced on the molecule by the hydration of the lipid head group leads to formation of the micelle. This type of micelle is known as a normal phase micelle (oil-in-water micelle).

Inverse micelles, on the other hand, include hydrophilic head regions positioned at the center of the micelle with the tails extending outwardly (water-in-oil micelle). Inverse (or reverse) micelles, with a hydrophilic core, are created using the microemulsion method. This type of micelle is specifically used to encapsulate hydrophilic materials. In a non-polar solvent, the exposure of the hydrophilic head groups to the surrounding solvent gives rise to a water-in-oil system. As a result, the hydrophilic groups are entrapped in the micelle core and the hydrophobic groups extend away from the center. Inverse micelles are generally smaller, tighter and more stable than regular micelles and liposomes.

A review of methods for producing liposome, micelles and inverse micelles is provided in Liposomes, Marc Ostro, ed., Marcel Dekker, Inc. New York, 1983, the relevant portions of which are incorporated herein by reference. See also Szoka, Jr. et al., (Ann. Rev. Biophys. Bioeng., 1980, 9:467), the relevant portions of which are also incorporated herein by reference.

The prolonged release of antibiotics is dependent, among other things, on the properties and sizes of the nanoparticles. A combination of various sizes of micelles, inverse micelles and liposomes (collectively, “nanoparticles’) is used herein to achieve the goal of prolonged release in view of the different half-life of each antibiotic. By manipulating the concentrations and sizes of the nanoparticles, controlled release of encapsulated antibiotics over time is achieved. The combination of inverse micelles and liposomes can be used, for example, for the encapsulation of any hydrophilic (water soluble) antibiotic such as vancomycin, gentamicin, gentamicin sulfate, tobramycin, ampicillin, penicillin, ethambutol, clindamycin, and a cephalosporin including cefazolin, ceftriaxone and cefotaxime for bacterial infections, acyclovir for viral infections, and amphotericin B for fungal infections. A combination of regular micelles and lipsosomes can be used for the encapsulation of hydrophobic antibiotics such as rifampicin, chloramphenicol, novobiocin, spectinomycin, trimethoprim (often supplied as a sulfamethoxazole), erythromycin, doxycycline and minocycline.

The present invention relates to a nanosystem capable of releasing drugs in a controlled manner using a combination of unilamellar and multilamellar liposomes along with regular and inverse micelles containing antibiotics. The alternating release times of these nanoparticles allow sustained antibiotic delivery over a specified time period. Liposomes and micelles are a completely biodegradable and non-toxic drug delivery system that has been extensively studied since 2000 for the ability to deliver therapeutic drugs. (Arkadiusz et al., 2000).

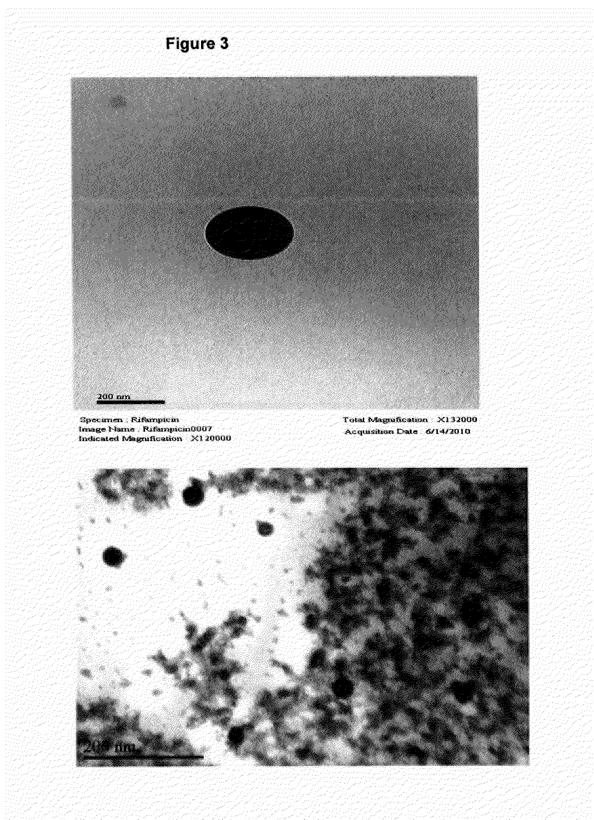

Unilamellar and multilamellar liposome vesicles, according to the present invention, are prepared using modified published methods such as reverse-phase evaporation and lipid hydration technique. (Mugabe et al., 2006a, Mugabe et al., 2006b, Otilia et al, 2005., Rawat et al, 2006). Referring to FIG. 3, rifampicin, a hydrophobic drug, is effectively encapsulated inside the nanoparticles. Various molar ratios of rifampicin and o-(decylphosphoryl)choline are first dissolved in methanol. The methanol is removed by rotary evaporation (45° C., 150 revolutions/min and 600 mm of Hg vacuum under a stream of Argon) to form a dry film. The film is rehydrated by vortexing for about 5 min and sonicating for about 5 min with about 0.01 mol/L acetate buffer (pH 5). The resulting aqueous dispersion is equilibrated in the dark for about 2 hours at about 25° C., and the excess drug is removed by centrifugation before characterization.

Double emulsion solvent extraction technique is also used to create drug delivery vehicles. PLGA (polylactic-co-glycolic acid) and 5% (w/v) polyethyleneglycol (PEG) is dissolved in about 2 ml of dichloromethane (DCM) separately. Suitable polymers generally include polyethylene glycol, polylactic and polyglycolic acids, and polylactic-polyglycolic and copolymers having a molecular weight between about 1,000-5,000 daltons. About 3 ml of rifampicin stock solution in PBS is measured using a drug to polymer ratio of 1:20. Both the drug and the polymer solutions are mixed with a high speed vortex mixer to form a stable emulsion. About 100 ml of 0.2% (w/v) aqueous polyvinylchloride solution is prepared by continuous stirring in moderate heat for about 1 hr. Afterwards the drug-polymer emulsion is poured into polyvinyl alcohol (PVA) solution which leads to the double emulsification of the particles. The mixture is sonicated for about 30 minutes and the particles are collected by centrifugation for about 15 minutes at about 13,000 rpm. The particles are washed with deionized water twice after the supernatant is discarded and are then resuspended in water and stored under refrigeration before Transmission Electron Microscopy (TEM) imaging as shown in FIG. 3. The upper scan shows encapsulated rifampicin nanoparticles. The lower scan shows no aggregation of the nanoparticles within the matrix.

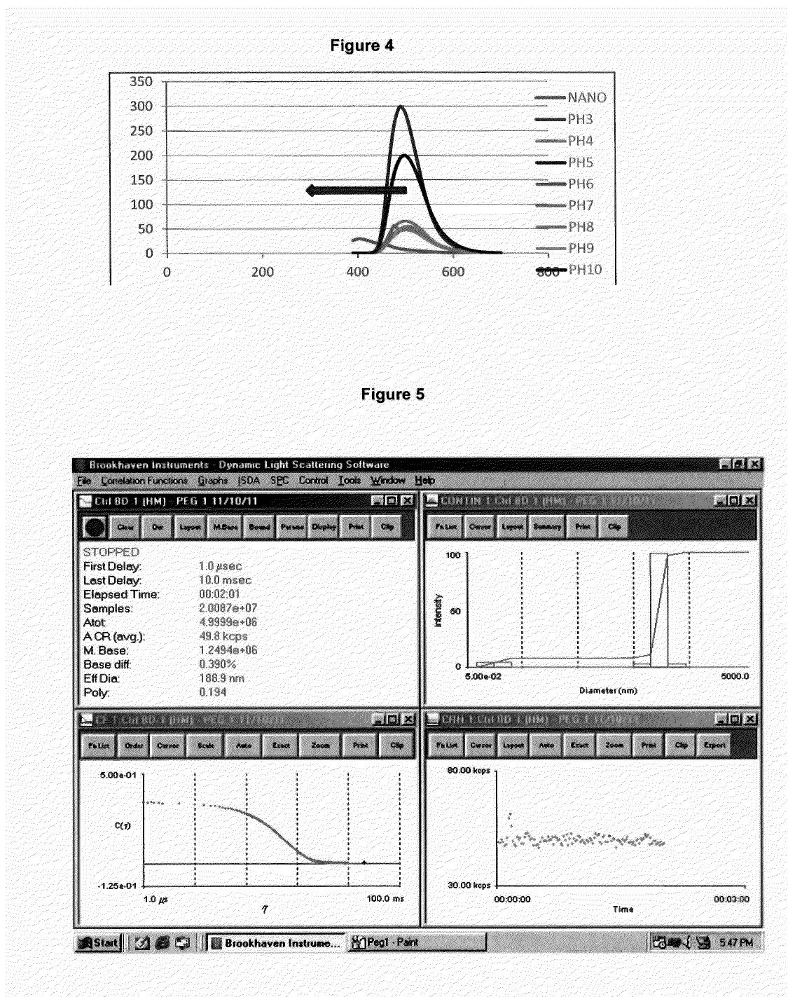

To ensure the drug rifampicin is indeed encapsulated within the vesicles, fluorescent spectroscopic analysis is conducted. FIG. 4 shows the fluorescent spectrum before (B-D) and after (A) the encapsulation. Not only did the fluorescent intensity dramatically decrease at the same concentration after the encapsulation, rifampicin nanoparticles also showed a blue shift (decreased wavelength) in the spectra which indicated the solvent environment had shifted from a hydrophobic environment to a more hydrophilic, polar environment. This data further supports encapsulation.

The chemical composition, total molecular weight and head/tail length ratios of micelle and liposomal monomers can be changed and modified in order to optimize the size, characterization and morphology. Moreover, this nanosystem can be customized according to the needs of the patient by varying entrapped antibiotics and the mixture of nanostructures. Finally, all nanovesicles in this system comprise organic materials, which are already used in many FDA approved drug delivery systems.

In order to provide antibiotic drug transport directly to the surgical site and, attain optimal nanoparticle stability, a polymer coating that contains antibiotic-encapsulated nanoparticles is applied over the body implant. First, a nanoparticulated drug-cocktail is mixed with a polymeric coating material and is then dissolved in a carrier solvent (commonly water or an alcohol). A thin film of nanoparticle-containing polymer is then brushed on the upper surface of the implant material which will set quickly using conventional UV light or chemical curing methods. When the carrier evaporates, the antibiotic-containing nanoparticles are stably attached to the surface providing sustained, localized release of the drug. Depending on the body implant, polymers suitable for use as coating materials according to the present invention include water-based polyvinylpyrrolidone, alcohol-based polymethylacrylate isobutene mono-isopropylmaleate, and hexamethyldisiloxane or isooctane solvent-based siloxane polymers.

Thus, as described herein, in one embodiment, the present invention relates to a pharmaceutical formulation comprising nanoparticles containing a therapeutically effective amount of at least one drug or antibiotic and a physiologically acceptable coating material whereby application of the formulation to an implant before surgery provides for extended release of the drug or antibiotic.

As used herein, a “therapeutically effective amount” of the antibiotic is an amount sufficient to provide the equivalent effect in a human of oral administration of the antibiotic in a range between about 1 mg/kg body weight and about 15 mg/kg body weight, more preferably between about 2 mg/kg body weight and about 10 mg/kg body weight. The amount of antibiotic used in the nanoparticles of the present application also depends on the unit area of application.

Examples of antibiotics suitable for use in ophthalmic devices include amoxicillin, sulfa drugs, erythromycin, streptomycin, tetracycline, clarithromycin, terconazole, azithromycin, bacitracin, ciprofloxacin, evofloxacin, ofloxacin, levofloxacin, moxfloxicin, gatifloxacin, aminoglycosides, tobramycin, gentamicin and polymyxin B combinations including polymyxin B/trimethoprim, polymyxin B/bacitracin and polymyxin B/neomycin/gramicidin, including pharmacologically acceptable salts and acids thereof.

Depending on the body implant, other suitable antibiotics include rifampicin, chloramphenicol, novobiocin, spectinomycin, trimethoprim, erythromycin, doxycycline, minocycline, vancomycin, acyclovir, amphotericin B, gentamicin, gentamicin sulfate, tobramycin, ampicillin, penicillin, ethambutol, clindamycin, and cephalosporins including cefazolin, ceftriaxone and cefotaxime, including pharmacologically acceptable salts and acids thereof.

In addition to antibiotics, other suitable drugs, as described herein, include steroids, anti-inflammatories, glaucoma treatment compounds, anti-histamines, dry eye medication, neuroprotectives, retinoids, antineovasculars, antioxidants and biologics.

Examples of steroids include glucocorticoids, aprogestins, amineralocorticoids and corticosteroids. Exemplary corticosteroids include cortisone, hydrocortisone, prednisone, prednisolone, methylprednisone, triamcinolone, fluoromethalone, dexamethasone, medrysone, betamethasone, loteprednol, fluocinolone, flumethasone, rimexolone and memetasone. Other examples of steroids include androgens, such as testosterone, methyltestosterone and danazol.

Examples of anti-inflamatories include NSAIDs such as piroxicam, aspirin, salsalate (Amigesic), diflunisal (Dolobid), ibuprofen (Motrin), ketoprofen (Orudis), nabumetone (Relafen), piroxicam (Feldene), naproxen (Aleve, Naprosyn), diclofenac (Voltaren), indomethacin (Indocin), sulindac (Clinoril), tolmetin (Tolectin), etodolac (Lodine), ketorolac (Toradol), oxaprozin (Daypro), and celecoxib (Celebrex).

Glaucoma treatment medications include beta-blockers, such as timolol, betaxolol, levobetaxolol, and carteolol; miotics, such as pilocarpine; carbonic anhydrase inhibitors, such as brinzolamide and dorzolamide; prostaglandins, such as travoprost, bimatoprost, and latanoprost; seretonergics; muscarinics; dopaminergic agonists; and adrenergic agonists, such as apraclonidine and brimonidine, and prostaglandins or prostaglandin analogs such as latanoprost, bimatoprost and travoprost.

Antihistamines and mast cell stabilizers include Olopatadine and epinastine, the acute care anti-allergenic products ketorolac tromethamine, ketotifen fumarate, loteprednol, epinastine HCl, emedastine difumarate, azelastine hydrochloride, Olopatadine hydrochloride, ketotifen fumarate; while the chronic care anti-allergenic products include pemirolast potassium, nedocromil sodium, lodoxamide tromethamine, cromolyn sodium.

Antineovasculars include biologics, Ranibizumab (Lucentis) and Bevacizumab (Avastin). Amblyopia medicine includes anesthetics and cycloplegics such as atropine. Dry eye medication includes cyclosporine.

Also, in a preferred embodiment, the physiologically acceptable coating material comprises a first component selected from the group consisting of polyvinylpyrrolidone, polymethylmethacrylate isobutene mono-isopropylmaleate, hexamethyldisiloxane and isooctane solvent-based siloxane polymers and copolymers thereof admixed with a second component selected from the group consisting of nitrocellulose, 2-octyl cyanoacrylate and n-butyl cyanoacrylate. More preferably, the physiologically acceptable coating material comprises polyvinylpyrrolidone as a first component admixed with nitrocellulose as a second component.

A method for the release of antibiotics from the implant over an extended period of time comprises providing an above-identified antibiotic-containing nanoparticle formulation and applying the formulation to the implant before surgery.

In another embodiment, a pharmaceutical formulation comprises first nanoparticles containing a therapeutically effective amount of a first antibiotic; second nanoparticles containing a therapeutically effective amount of a second antibiotic; and a physiologically acceptable coating material. Application of the formulation to an implant before surgery provides for extended release of the first and second antibiotics to treat infection.

The first antibiotic can be hydrophobic and is selected from the group consisting of rifampicin, chloramphenicol, novobiocin, spectinomycin, trimethoprim, erythromycin, doxycycline and minocycline, including pharmacologically acceptable salts and acids thereof. The second antibiotic can be hydrophilic and is selected from the group consisting of vancomycin, acyclovir, amphotericin B, gentamicin, gentamicin sulfate, tobramycin, ampicillin, penicillin, ethambutol, clindamycin and cephalosporins including cefazolin, ceftriaxone and cefotaxime, including pharmacologically acceptable salts and acids thereof. In the alternative, both antibiotics can be hydrophobic or both antibiotics can be hydrophilic.

In a preferred embodiment, the corresponding method provides for the extended release of antibiotics from the implant comprising providing an above-identified first and second antibiotic-containing nanoparticle formulation and applying the formulation to the implant before surgery. This specific targeting drug delivery system can help reduce dangerous side effects. It also eliminates the time that is otherwise needed for the drugs to be processed by the liver. Therefore, a lesser amount of drug will have the same beneficiary effects compared with the drugs being administered intravenously or orally.

For example, clindamycin is effective against both aerobic and anaerobic bacterial infections. Usually clindamycin is administrated orally, absorbed through the gastrointestinal tract, extensively metabolized in the liver, and then distributed throughout the body. Only a small therapeutic concentration (between 5 and 10 percent) can be transmitted to the brain after 1.5 to 5 hours after administration of the drug. Since it has to be systematically circulated, a much higher initial dose is required for the effective dosage to reach the brain. A higher initial dosage leads to more severe side effects such as headache, bloody diarrhea, fever, nausea, severe blistering of the skin and jaundice which all can be reduced to a minimum by administrating the effective dosage directly to the infected area according to the present invention.

### Materials and Methods

The coating methodology of the present invention is applicable to at least the following implants:

### Ocular Implants (for the Anterior Portion of the Eye)

Cataracts are a common condition in the elderly. By age 75, more than half the people in the United States either have a cataract or have undergone cataract surgery. The only effective treatment for cataracts is surgical removal of the cloudy, damaged lens and replacing it with an artificial lens—an intraocular lens (IOL) implantation. However, severe post-surgical infections including inflation of the lens, glaucoma (increase in eye pressure) and bacterial endophthalmitis intraocular infection are among the complications of cataract surgery. Currently, post cataract surgery management requires the use of topical antibiotics such as fluoroquinolone to prevent bacterial intraocular infections. However, topical application of antibiotics has a very low level of intraocular penetration (about 0.3%). Thus, a higher effective concentration of drugs is needed in the topical treatment of intraocular infections. Moreover, topical treatment is not only costly; but it can be toxic to the ocular surface.

The present invention relates to a unique drug delivery system which is capable of providing direct drug transport to the lens and retina of the eye. The invention includes three major components: (1) the encapsulation of hydrophobic antibiotics (for example, fluoroquinolone, chloramphenicol and/or rifampicin) and hydrophilic antibiotics (for example, vancomycin and acyclovir) into nanoparticles; (2) the incorporation of antibiotic nanoparticles (from step (1)) into a polymeric, non-toxic coating material (Poloxamer, hydroxypropylmethylcellulose, methylcellulose, polyvinyl alcohol, and polyvinyl pyrrolidone) with a volatile carrier solvent (for example, ethyl acetate or ethanol); and (3) the direct application of product from steps (1) and (2) to IOL implants prior to surgery. The IOL implants can comprise a (hard) polymethyl methacrylate lens or any of the commercially available foldable lenses comprising silicone and acrylic materials well known to those skilled in the art.

When the volatile carrier solvent evaporates, the coating polymer with embedded antibiotic nanoparticles forms a thin film that is capably attached to the surface of the implant material. Local application of encapsulated antibiotics directly to the surgical sites provides a non-oral, non-intravenous, controlled time-release treatment, which allows continuous administration of antibiotic therapy over the prescribed time span of the individual antibiotics used. The present invention provides a novel chemotherapeutic regime for the prevention and treatment of bacterial, fungal and viral infections which often occur in IOL implant patients with a more efficient effective dose.

### Methods

### Step 1: Encapsulation

### Hydrophobic Antibiotic Nanoparticles

An organic phase is formed by dissolving the hydrophobic anti-bacterial drug chloramphenicol in about 5 ml of ethyl acetate with o-decylphosphoryl choline (4:1 drug to surfactant ratio) followed by heating to about 50° C. In the alternative, other surfactants including Surfynol 465, PLGA-PEG copolymer and L-α-phosphaditylcholine can be used.

The resulting organic phase is titrated into a water phase of about 5 ml which contains a 4:1 drug to polymer ratio of either high molecular weight polyethylene glycol (HW PEG), polyvinyl pyrrolidone (PVP) or poloxamer. In lieu of HW PEG and poloxamer, PVA (polyvinyl alcohol) can be used in the water phase. All three polymers are non-toxic to the eyes and work well to form nanoparticles.

The organic phase is added to water phase under constant high sheer stirring while the water phase is maintained at about 50° C. Micelles are formed in this oil-in-water (o/w) emulsion. The solution is heated to about 77° C. (the boiling point of ethyl acetate) with constant stirring in high sheer until all of the ethyl acetate from the organic phase is evaporated. By reducing the final volume to about 5 ml (the original volume of the water phase), the formation of nanoparticles is confirmed.

The sizes of the nanoparticles are characterized using TEM Dynamic Light Scattering (DLS).

Referring to FIG. 5, the DLS data in the upper left hand box shows a nanoparticle average size of about 188.9 nm. Good poly-dispersity constant (0.194, <20%) indicates a uniform size distribution. All the nanoparticles have the same decay constant which indicates there is no contamination.

Unilamellar Liposome Water-Oil-Water (w/o/w) Emulsion for Hydrophilic Drug

The primary oily phase is prepared by mixing about 5 ml of castor oil with either about 0.02 g of L-α-phosphaditylcholine or about 1 ml of Surfynol 465.

Either a luminescence marker or a hydrophilic drug is dissolved in about 5 ml water with about 1.0 g of HW PEG to form a water phase.

The water phase is titrated into the primary oil phase with constant stirring under low heat (about 60° C.).

The mixture is sonicated for about 1 hour then maintained for about 24 hours. Any remaining fluid is discarded, and a w/o emulsion is formed.

The final aqueous phase (about 20 ml of de-ionized water with about 2.0 g PVA or poloxamer) is heated to about 50° C. The w/o emulsion is titrated into the final aqueous solution and is sonicated for about one hour under high sheer to create the unilamellar liposome. The final product is a water-oil-water emulsion.

Reverse Micelles (Water-in-Oil (w/o) Emulsion) for Hydrophilic Drug

About 2.0 ml of lecithin is mixed with about 5 ml of vitamin E (vitamin F and castor oil can also be used) with gentle heating (about 60° C.) and continuous stirring until the lecithin is dissolved. The water phase comprises about 2 ml of water, about 2.0 ml of low molecular weight PEG and the drug, and is heated to about 50° C.

The water phase is added dropwise with constant high sheer stirring into the oil phase. The mixture is then sonicated for about 15 min. The final w/o emulsion is maintained for about 24 hours with constant low sheer stirring. Any remaining aqueous layer is discarded.

### Step 2: Coating Material

About 2.0 g of Hypromellose (hydroxypropyl methylcellulose or HPMC) is dissolved in about 5.0 ml of ethyl acetate with constant stirring under low heat (about 65° C.). Hypromellose is a semisynthetic, inert, viscoelastic polymer that relieves dryness and eye irritation caused by reduced tear flow and is used as a surgical aid for cataract removal and lens implantation procedures. HPMC also helps maintain the shape of the eye during surgery and protects the eye from damage.

After the Hypromellose is totally dissolved, about 0.2 g of polycaprolactone is added to the solution, and temperature is maintained at about 65° C. Ethyl acetate is added dropwise, if necessary, until the polymer is totally dissolved. The resulting solution is cooled to about 35° C., and the antibiotic encapsulated nanoparticles, liposomes and reverse micelles are added to the coating material. The product is cooled to about 25° C. before storage or application to an implant.

FDA approved materials are used in order to avoid the extended governmental approval and evaluation process. Thus, the final product can be made available to consumers as soon as reasonably possible. Hypromellose (hydroxypropyl methylcellulose) is used as the primary ingredient in the coating material and has been used in many ophthalmic applications. Hypromellose exhibits a thermal gelation property in aqueous solution. That is, when the solution is heated to a critical temperature, the solution congeals into a non-flowing but semi-flexible mass. It also functions as a controlled-release agent to delay the release of a medicinal compound. An important factor is Hypromellose is biodegradable within the eyes and it has been proven that its decomposition products are actually beneficial to the eyes as a lubricant. Therefore, after 8 weeks, when the intended medication is totally delivered, Hypromellose is also totally degraded and utilized. The implant remains as it was before coating and the risk of acquiring post-surgical infections is reduced.

### Step 3: Sustained Release Study

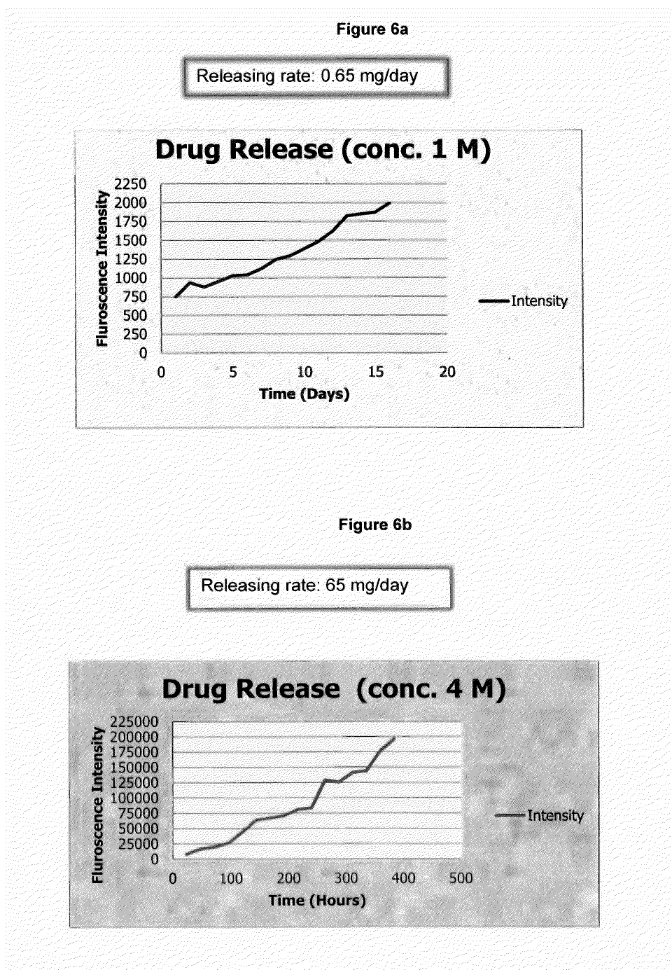

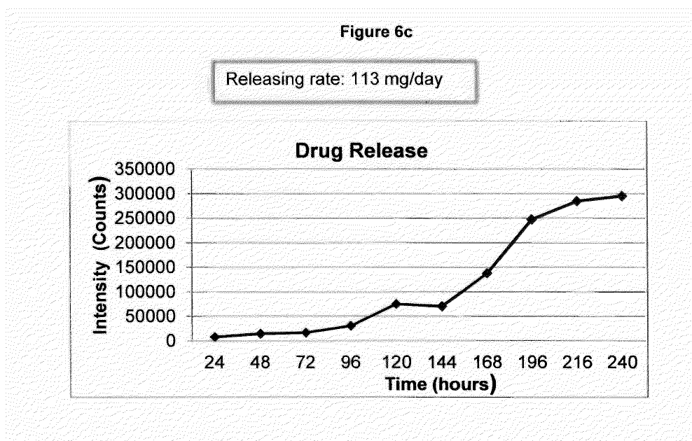

FIGS. 6a and 6b show the sustained release of chloramphenicol (at 1 and 4 molar concentrations, respectively) from the liposomes described above as a function of fluorescence intensity vs. time at a release rate of about 0.65 mg/day.

FIG. 6c shows the sustained release of vitamin E from the reverse micelles described above as a function of intensity vs. time at a release rate of about 113 mg/day.

### Shunt Device

Glaucoma is the leading cause of irreversible blindness worldwide and the second leading cause of blindness, other than cataracts. There is a strong correlation between high intraocular pressure and glaucoma. In glaucoma patients, the aqueous humor builds up within the eye producing increased pressure. Glaucoma ocular implants or shunt devices are used to continuously decompress elevated intraocular pressure in eyes. These devices divert excess aqueous humor from the anterior chamber of the eye into the Schlemm's canal where post-operative patency can be maintained. These devices are implanted in the eye to provide an artificial alternative drainage site for fluid to exit the eye. The coating material of the present invention can be applied to such a device to reduce or minimize post-surgical complications.

### Ocular Implants (for the Posterior Portion of the Eye)

Unlike cataracts and glaucoma, there are several diseases such as age-related macular degeneration (AMD) and retinitis pigmentosa which occur at the posterior segment of the eye. Macromolecules of about the same size as antibodies are unlikely to penetrate the internal limiting membrane. A systemic administration approach is an effective means for overcoming the problem of the blood-retinal barrier. However, the requirement of large doses of the drug produces undesirable side effects. Currently there are several types of posterior implants for the eyes that are suitable for drug delivery to the vitreous chamber of the eye.

### Intrascleral Implant (ISI)

The present invention is also beneficial for use with intrascleral implants. However, there currently are no commercially available intrascleral implants on the market for human use.

### Implantable Miniature Telescope (IMT)

VisionCare, Inc, an Israeli company, has developed an Implantable Miniature Telescope (IMT), which may be a permanent solution for vision loss due to age-related macular degeneration (AMD). On Jul. 6, 2010, the FDA announced approval of the Implantable Miniature Telescope (IMT) for improving vision in certain patients with end-stage age-related macular degeneration (AMD).

An IMT has a similar look and circular dimension to an IOL, but has greater depth. Surgically implanted in one eye, the IMT is a micro-sized precision telescope that replaces the natural lens and provides an image that is magnified more than two times. AMD, a condition that mainly affects older people, damages the center of the retina (macula) and results in a loss of vision in the center of the visual field. About 8 million people in the United States have AMD and nearly 2 million of those individuals already have significant vision loss, according to the National Eye Institute.

By coating the surrounding surface (of the IMT) which comes into contact with eye tissue, the present invention can not only deliver antibiotics to prevent post-surgical infections, but can also deliver an antioxidant regiment and anti-VEGF medications such as Avastin® (bevacizumab), Lucentis (ranibizumab) and Macugen (pegaptanib) which are commonly injected directly into the rear portion of the eye. Antioxidants and Anti-VEGF medication work together to delay the vision loss linked to wet AMD thus preventing the total loss of vision of implant patients.

### Dental Implants

Root form implants are the closest in shape and size to the natural tooth root. They are commonly used in wide, deep bone to provide a base for replacement of one, several or a complete arch of teeth. The bone and gums surrounding a dental implant can become infected due to biofilm formation. A certain proportion of implants is not successful and may fail due to infection. Infected implants are colonized by subgingival species including Porphyromonas gingivalis, Bacteroides forsythus, Fusobacterium nucleatum, Campylobacter gracilis, Streptococcus intermedius and Peptostreptococcus micros. The present invention can prevent dental biofilm from forming by the application of an antibiotic nanoparticle embedded coating prior to oral surgery.

### Cochlear Implants (IC)

A cochlear implant (CI) is a surgically implanted electronic device that provides a sense of sound to a person who is profoundly deaf or severely hard of hearing. Cochlear implants are often referred to as a bionic ear. As of December 2010, approximately 219,000 people worldwide have received cochlear implants. In the U.S., roughly 42,600 adults and 28,400 children are recipients. The solid surface of a cochlear implant can be coated with an antibiotic nanoparticle embedded coating to prevent post-surgical infections.

### Nasal Implants

Dorsal, Columella and Premaxilla Synthetic Implants (nasal augmentation) are used in reconstruction and cosmetic procedures. Nasal implants are utilized to correct deficiencies in the dorsal area (bridge) of the nose and the premaxilla (base). Sometimes implants are used to correct cartilage deficiencies or to reconstruct after trauma. There are several types of materials used for nasal implants which are compatible with the present coating material: silicone implants, expanded polytetrafluoroethylene (ePTFE) implants, polyethylene implants and hydroxyapatite implants.

### Hip and Shoulder Replacement Implants

Numerous orthopedic manufacturing companies produce different implants for use in hip and shoulder replacement surgery. Most of these companies make several different replacement prostheses. Most of the materials that are used to make these implants such as titanium, stainless steel, cobalt chrome, polyethylene plastic and ceramics are all compatible with the coating material of the present invention.

### Knee Replacement Implants

A replacement knee joint comprises a flat metal plate and stem implanted in a tibia of an individual, a polyethylene bearing surface and a contoured metal implant fit around the end of the femur. The use of components made from metals and polyethylene allow for optimum articulation (or joint mobility) between the joint surfaces with minimal wear. Because the knee implant has a flatter bearing, wear is less of a problem than in a hip implant which has a very deep bearing.

Materials which can be used in knee implants and are also compatible with the coating materials of the present invention include: 1) stainless steel; 2) cobalt-chromium alloys; 3) titanium and titanium alloys; 4) un-cemented implants wherein surface of the titanium is modified by coating the implant with hydroxyapatite; 5) tantalum; 6) polyethylene plastic and 7) zirconium alloys.

### REFERENCES

Arkadiusz K, Gubernator J, Przeworska E, and Stasiuk M. “Liposomal drug delivery, a novel approach: PLARosomes.” Acta Biochim. Polo., 2000, 47, 639-649.
Garay-Jimenez J C; Gergeres D; Young A; Lim D V; Turos E. “Physical properties and biological activity of poly(butyl acrylate-styrene) nanoparticle emulsions prepared with conventional and polymerizable surfactants.” Nanomedicine: Nanotechnology, Biology, and Medicine (Nanomedicine), 2009: 5(4): 443-51.
Henry C M. “Cover Story; Drug Delivery” C&E News Washington, 2002, 80, 39-47.
Johanson C E, Duncan J A, Stopa E G, Baird A. “Enhanced Prospects for Drug Delivery and Brain Targeting by the Choroid Plexus-CSF Route.” Pharm. Res., 2005 July, 22(7):1011-37.
Mugabe C, Halwani M, Azghani A O, Lafrenie R M, Omri A. “Mechanism of Enhanced Activity of Liposome-Entrapped Aminoglycosides against Resistant Strains of Pseudomonas aeruginosa.” Antimicrobial Agents and Chemother., 2006, 50, 2016-2022.
Mugabe C, Azghani A O, Omri A. “Preparation and Characterization of Dehydration-rehydration vesicles loaded with aminoglycosides and microlide antibiotics.” Int. J Pharm., 2006, 307, 244-250.
Müller-Goymann CC. “Physiochemical characterization of colloidal drug delivery system such as reverse micelles, vesicles, liquid crystals and nanoparticles for topical administration.” European Journal of Pharmaceutics and Biopharmaceutics, 2004, 58, 343-356.
Nandi I, Bari M, Joshi H. “Study of Isopropyl Myristate Microemulsion Systems Containing Cyclodextrins to Improve the Solubility of 2 Model Hydrophobic Drugs.” 2003, 4, 1-9.
Koo O M, Rubinstein I, Onyuksel H. “Camptothecin in sterically stabilized phospholipid micelles: A novel nanomedicine.” Nanomedicine: Nanotechnology, Biology and Medicine, 1 (2005) 77-84.
Rawat M., Singh D., Saraf S. “Nanocarriers: Promising Vehicle for Bioactive Drugs.” Biol. Pharm. Bull., 29(9) (2006) 1790-1798.
Richter A, Olbrich C, Krause M, Hoffmann J, Kissel T. “Polymeric Micelles for parenteral delivery of Sagopilone: Physicochemical characterization, novel formulation approaches and their toxicity assessment in vitro as well as in vivo.” European Journal of Pharmaceutics and Biopharmaceutics, 2010: 75, 80-9.
Torchilin VP. “Structure and design of polymeric surfactant-based drug delivery system.” Journal of Control Release, 2001, 73, 137-172.
Wang C H, Wang W T, Hsiue G H. “Development of polyion complex micelles for encapsulating and delivering amphotericin B.” Biomaterials, 2009: 30(19): 3352-8.

Although the present invention has been disclosed with respect to particular nanoparticles, antibiotics, formulations and methods, it will be apparent that a variety of modifications and changes can be made without departing from the scope and spirit of the invention, as described and claimed herein.

What is claimed is:

1. A composition comprising nanoparticles containing a therapeutically effective amount of at least one drug and a physiologically acceptable coating material whereby application of the composition to a body implant provides for extended release of the drug to treat a physiological condition.

2. The composition according to claim 1 wherein the implant comprises an ophthalmic device.

3. The composition according to claim 1 wherein the drug comprises an antibiotic selected from the group consisting of fluoroquinolone, chloramphenicol, rifampicin, vancomycin and acyclovir, including pharmacologically acceptable salts and acids thereof.

4. The composition according to claim 1 wherein the drug is selected from the group consisting of an antibiotic, a steroid, an anti-inflammatory agent, a glaucoma treatment compound, an antihistamine, a dry eye medication, a neuroprotective agent, an antineovascular agent and an antioxidant.

5. The composition according to claim 1 wherein the physiologically acceptable coating material comprises a first component selected from the group consisting of poloxamer, hydroxypropylmethyl cellulose, methylcellulose, polyvinyl alcohol and polyvinyl pyrrolidone and a second component including polycaprolactone.

6. The composition according to claim 1 wherein the implant comprises a dental implant, a cochlear implant, a nasal implant, a vascular graft, a stent and a hip, shoulder or knee replacement device.

7. The composition according to claim 1 wherein the drug comprises an antibiotic selected from the group consisting of rifampicin, chloramphenicol, novobiocin, spectinomycin, trimethoprim, erythromycin, doxycycline, minocycline, vancomycin, acyclovir, amphotericin B, gentamicin, gentamicin sulfate, tobramycin, ampicillin, penicillin, ethambutol, clindamycin, and cephalosporins including cefazolin, ceftriaxone and cefotaxime, including pharmacologically acceptable salts and acids thereof.

8. The composition according to claim 1 wherein the implant is formed of a material selected from the group consisting of polymethyl methalcrylate, hydroxyapatite, hydrogel, silicone, polytetrafluoroethylene, polyethylene, titanium, stainless steel, cobalt-chromium alloys, ceramic, titanium alloys, tantalum and zirconium alloys.

9. The composition according to claim 1 wherein the physiologically acceptable coating material comprises a first component selected from the group consisting of polyvinylpyrrolidone, polymethylmethacrylate isobutene mono-isopropylmaleate, hexamethyldisiloxane and isooctane solvent-based siloxane polymers and copolymers thereof admixed with a second component selected from the group consisting of nitrocellulose, 2-octyl cyanoacrylate and n-butyl cyanoacrylate.

10. A method for the sustained release of a drug from a physiologically acceptable coating material applied to a body implant comprising:
a) encapsulating the drug into nanoparticles;
b) incorporating the nanoparticles into the physiologically acceptable coating material to form a nanoparticle-embedded polymeric coating material; and
c) applying the product of step b to the implant before surgery
whereby the drug is released from the implants over an extended period of time to treat a physiological condition.

11. The method according to claim 10 wherein the implant comprises an ophthalmic device.

12. The method according to claim 10 wherein the drug comprises an antibiotic selected from the group consisting of fluoroquinolone, chloramphenicol, rifampicin, vancomycin and acyclovir, including pharmacologically acceptable salts and acids thereof.

13. The method according to claim 10 wherein the drug is selected from the group consisting of an antibiotic, a steroid, an anti-inflammatory agent, a glaucoma treatment compound, an antihistamine, a dry eye medication, a neuroprotective agent, an antineovascular agent and an antioxidant.

14. The method according to claim 10 wherein the physiologically acceptable coating material comprises a first component selected from the group consisting of poloxamer, hydroxypropylmethyl cellulose, methylcellulose, polyvinyl alcohol and polyvinyl pyrrolidone and a second component including polycaprolactone.

15. The method according to claim 10 wherein the implant comprises a dental implant, a cochlear implant, a nasal implant, a vascular graft, a stent and a hip, shoulder or knee replacement device.

16. The method according to claim 10 wherein the drug comprises an antibiotic selected from the group consisting of rifampicin, chloramphenicol, novobiocin, spectinomycin, trimethoprim, erythromycin, doxycycline, minocycline, vancomycin, acyclovir, amphotericin B, gentamicin, gentamicin sulfate, tobramycin, ampicillin, penicillin, ethambutol, clindamycin, and cephalosporins including cefazolin, ceftriaxone and cefotaxime, including pharmacologically acceptable salts and acids thereof.

17. The method according to claim 10 wherein the implant is formed of a material selected from the group consisting of polymethyl methalcrylate, hydroxyapatite, hydrogel, silicone, polytetrafluoroethylene, polyethylene, titanium, stainless steel, cobalt-chromium alloys, ceramic, titanium alloys, tantalum and zirconium alloys.

18. The method according to claim 10 wherein the physiologically acceptable coating material comprises a first component selected from the group consisting of polyvinylpyrrolidone, polymethylmethacrylate isobutene mono-isopropylmaleate, hexamethyldisiloxane and isooctane solvent-based siloxane polymers and copolymers thereof admixed with a second component selected from the group consisting of nitrocellulose, 2-octyl cyanoacrylate and n-butyl cyanoacrylate.
